(19) United States
(12) Patent Application Publication
(10) Pub. No.: US 2026/0179773 A1
(43) Pub. Date: Jun. 25, 2026

(54) SYSTEM FOR DECISION-MAKING SUPPORT UTILIZING MULTI-ATTRIBUTE TRADESPACE EXPLORATION AND CLINICAL COST-EFFECTIVENESS ANALYSIS

(71) Applicant: Daniel Herndon Richards, Malden, MA (US)

(72) Inventor: Daniel Herndon Richards, Malden, MA (US)

(21) Appl. No.: 19/424,463

(22) Filed: Dec. 18, 2025

Related U.S. Application Data
(60) Provisional application No. 63/736,083, filed on Dec. 19, 2024.
(60) Provisional application No. 63/816,235, filed on Jun. 2, 2025.

(51) Int. Cl. G16H 50/20; G06Q 30/0201; G16H 20/00

(52) U.S. Cl. CPC G16H 50/20; G16H 20/00; G06Q 30/0206

(57) ABSTRACT

A system may model, in a computer system, a first model representing a plurality of decision-options. A system may model, in the computer system, a second model representing an effectiveness of the plurality of decision-options. A system evaluates sensitivities, uncertainties, and alternative scenarios related to the plurality of decision-options of the first model and the effectiveness of the plurality of decision-options of the second model. A system provides an output indicating recommendations and tradeoffs among the set of design options selected from the plurality of decision-options. A system and method for decision-making support in medical technology strategy integrates Multi-Attribute Tradespace Exploration (MATE) and Clinical Cost-Effectiveness Analysis (CEA) models. The system models a plurality of decision-options, and evaluates their effectiveness using clinical effectiveness metrics such as quality-adjusted life years (QALYs). The system optimizes these decision-options by performing probabilistic analyses, including Monte Carlo simulations, to account for uncertainties.

### CROSS-REFERENCE TO RELATED APPLICATIONS

This application claims the benefit of priority under 35 U.S.C. § 119(e) to U.S. Provisional Patent Application Ser. No. 63/816,235, filed Jun. 2, 2025, entitled “SYSTEM FOR DECISION-MAKING SUPPORT UTILIZING MULTI-ATTRIBUTE TRADESPACE EXPLORATION AND CLINICAL COST-EFFECTIVENESS ANALYSIS” and claims the benefit of priority under 35 U.S.C. § 119(e) to U.S. Provisional Application Ser. No. 63/736,083, filed Dec. 19, 2024, entitled “SYSTEM FOR DECISION-MAKING SUPPORT UTILIZING MULTI-ATTRIBUTE TRADESPACE EXPLORATION AND CLINICAL COST-EFFECTIVENESS ANALYSIS”, each of which is incorporated by reference herein in its entirety.

### BACKGROUND

There are many different approaches to designing and evaluating medical systems and treatments. Because these systems and factors in their design are complex, significant time, investment and effort is needed to perfect the quality of these systems and treatments.

### SUMMARY

In some aspects, some embodiments relate to combining elements of Multi-Attribute Tradespace Exploration (MATE) and Clinical Cost-Effectiveness Analysis (CEA) approaches to create a decision-making support framework specifically for medical technology (MedTech) system architects. This combined model integrates the system architectural decision-options of MATE with the clinical effectiveness metrics of CEA, such as quality-adjusted life years (QALYs), revealing a system architecture and first indication with superior clinical and reimbursement potential.

While MATE is traditionally used in system architecting practices to weigh trade-offs in decision-options based on intermediate performance metrics (e.g., size, weight, serviceability), project-level metrics of value delivery (e.g., cost, development time, risk), and abstract measures of stakeholder ‘Utility’, aspects described herein introduce the use of clinical effectiveness as a key metric in the tradespace plot. This approach is novel because the approach (according to some embodiments) posits relationships between the function of the technology (as defined or modified by the decision-option) and its known or anticipated impact on higher-order health-related outcomes like clinical effectiveness, which is not done in existing practices. In some embodiments, systems and methods are provided that permit a person guiding the process to posit relationships between decision alternatives (alternative decision-options) and the impact of each option on clinical effectiveness. This is especially useful when the measure of health-related outcomes (e.g., in QALYs) is multifactorial and incorporates any number and combination of clinical reported outcomes, clinical risks, treatment side effects, patient reported outcomes, etc.

Additionally, in some implementations, the system leverages probabilistic analysis and Monte Carlo simulations to account for uncertainty in the relationships posited between decision alternatives and the impact of each option on health-related outcomes (e.g., CEA). In some aspects of some implementations, the analysis is extended to include uncertainties related to potentially accompanying contributors to health-related outcomes, too, for example uncertainties in related (known or speculative) clinical research, related (known or speculative) outcome measurement techniques, alternative clinical development strategies, related (known or speculative) diagnostics, related (known or speculative) clinical practices, trends in clinical decision situations, market disruptions, or value-enhancing technologies like drug delivery technologies, digital health, or precision medicine. Accordingly, extending the analysis to include all sources of uncertainty (e.g., technological and non-technological, clinical and non-clinical, component and aggregate, known and anticipated, etc.) further enhances the strategic decision-making process. It is known that uncertainty analyses may be conducted within analyses for portfolio management purposes (e.g., in a net-present value (NPV) analysis for each project or investment), and, therefore, this method that incorporates all relevant sources of uncertainty (as with combining MATE and CEA, for example) in such a way that enables exhaustive and holistic modeling of all potential sources of (and threats to) value (and their uncertainties) provides a unique and useful framework for evaluating medical technology strategies, making it a novel contribution to the field.

Some aspects relate to a method and system that presents tradeoffs in the financing (as with investment), development (as with research and development), or deployment (as with treatment) of any product or service that may be used to measure and/or improve human and/or animal health (MedTech). More specifically, certain embodiments relate to a method and system that presents an n-dimensional (1 or more dimensions) plot of decision-options, where one or more axes represents the performance of one or more decision-options in terms of the associated clinical effectiveness (or derivative metrics and analyses like clinical cost-effectiveness, incremental cost-effectiveness ratio (ICER), willingness-to-pay (WTP), etc.). More specifically, each plotted decision-option can represent a combination of decision-options as is done in the practice of Multi-Attribute Tradespace Exploration (MATE), which can be used for architecting systems, processes, organizations, or any designed system.

It is appreciated that while MATE is used in MedTech research and development, intermediate measures of value are often used such as cost, development time, physical envelope, weight, usability, etc., and higher-order measures of value are reported as “utility” (and/or multi-attribute utility), rather than using overarching higher-order health-related KPIs like clinical effectiveness. Clinical effectiveness modeling (to produce estimates of clinical effectiveness or clinical cost-effectiveness) is used as a measure of “value” in strategic, clinical, and reimbursement decision-making in MedTech. Additionally, it is appreciated that “value” considers other health-related metrics, such as Equity, Value of Hope, and Reduction in Uncertainty, to name a few from the ISPOR “value flower”, which is widely used in health economics and outcomes research (HEOR). In some aspects, some embodiments relate to a system that allows side-by-side comparison of decision-options in terms of their performance against higher-order health-related KPIs. By combining the practices of MATE with the practices of calculating health-related metrics (and their uncertainties), some aspects described herein relate to a method and system that is novel and useful in MedTech.

Some aspects of consistent with principles of the invention relate to a method and system that results in relationships posited between decision-options and their impact within a clinical effectiveness model. While the positing of relationships between system decision-options and performance may be common for a MATE model, it is novel for a system or method to establish a relationship between system development and/or investment decision-options and their impact within a clinical effectiveness model. Although it is known that Clinical Cost-Effectiveness Analysis (CEA) is used in MedTech strategy to indicate potential clinical value of clinical development (treatment) alternatives, it is novel to represent in CEA of treatment alternatives as both clinical development alternatives and their uncertainties with system design alternatives.

In some aspects, some embodiments relate to an integration of a Multi-Attribute Tradespace Exploration (MATE) model and a Clinical Cost-Effectiveness Analysis (CEA) model to create a decision-making support framework specifically for medical technology strategists. This combined model uniquely incorporates the architectural decision-options of a MATE. In some aspects, some embodiments relate to combining elements of Multi-Attribute Tradespace Exploration (MATE) and approaches to estimate of health-related outcomes, such as Clinical Cost-Effectiveness Analysis (CEA), to create a decision-making support framework specifically for medical technology strategists. This combined model integrates the architectural decision-options of MATE with the patient health-related outcome metrics such as clinical effectiveness (as with QALYs calculated in CEA), health equity, value of hope, reduction in uncertainty, and others. Such capabilities may be implemented in a computer-implemented system and/or apparatuses that provides decision support functions to one or more computer users. The system/apparatus may perform one or more acts relating to models specified by users to determine one or more recommended design options.

Some embodiments present health-related outcomes (inclusive to clinical effectiveness and derivative metrics ICER and analyses like WTP) on one or more axes and other metrics (e.g., physical attributes, performance, regulatory risk, cost, user preference, QALYs, equity, utility, and multi-attribute utility, among others) on other axes in the n-dimensional plot. It is also appreciated that “axes” can refer to any readout where the decision-options are indicated, which is inclusive to varying the appearance of the indicated decision-option (and/or it callout), such as through color, size, shape, list order, or symbology (including text), among others, so that performance of the option against any number of metrics is indicated or codified into a data set for further analysis.

It is also appreciated that insights may be realized from some embodiments, such as:
The conditions under which one treatment may be selected over any number of other treatments to treat patients in any number of clinical decision situations.
The conditions under which one treatment may be reimbursable in the context of any number of other alternative treatments available in any number of clinical decision situations.
The potential value (upside potential) of pursuing clinical evidence of an anticipated relative benefit (or risk) of one decision-option relative to another, to an existing solution within the standard of care, or to an imagined competing alternative, now and in the future, for example.
Through probabilistic sensitivity analysis, the main sources of uncertainty that impact higher-order health-related outcomes like clinical cost-effectiveness, including uncertainty in technology function, uncertainty in clinical development, uncertainty in the future landscape, uncertainty in user preferences, and more.
Through system architecture best practices (especially effective decision-option scoping), an exhaustive evaluation of the universe of decision-options (and combinations of decision-options), especially by establishing mutually exclusive and collectively exhaustive (MECE) decision-options that conceptually constrain the universe of decision-options, in terms their performance against any number of metrics of strategic interest, including any meaningful inputs to development planning (e.g., development risk, regulatory risk, development cost, time to value, etc.), as well as tradeoffs with other metrics.
A probabilistic, practical definition of Utopia as the maximum attainable “value” (especially as driven by health-related outcomes) for any number of patient populations in any number of clinical decision situations.
A set of potentially attractive strategies, including the conditions under which the set of strategies would narrow to fewer strategies.
All of the above insights with all variables and distributions replaced with forecasted values to project insights into the future, with associated uncertainties to accommodate uncertainty in forecasts.
Inclusive to all the above insights, a holistic sense of the strategic landscape applicable in present terms, as well as applicable upon future model updates when new data become available or in “real-time” as with a living “digital twin” model.

Some key aspects include:
Integration of Clinical Effectiveness Analysis (CEA): While MATE is traditionally used to weigh trade-offs in decision-options based on intermediate metrics like cost, development time, size, weight, and reliability, some embodiments described herein uses clinical effectiveness as a key metric in the tradespace plot. In some aspects of some embodiments, this approach posits relationships between the function of the technology and its impact on clinical effectiveness, which is not done in existing design practices.
Probabilistic Analysis and Monte Carlo Simulations: In some aspects, some embodiments leverage probabilistic analysis and Monte Carlo simulations to account for uncertainty in the outcomes, further enhancing the decision-making process. This integration of uncertainty modeling with MATE and CEA provides a comprehensive framework for robustly evaluating tradeoffs in medical technology strategies, among other health-related problem spaces.
Combined MATE and CEA Model: In some embodiments, the system combines the—MATE and CEA models into a single framework, allowing for evaluation of medical technology strategies with consideration of their ultimate clinical performance. This combined model supports decision-making by providing a coordinated visualization of development and investment decision-options in terms of multiple dimensions of value, their uncertainty, and the attributes of other treatment options.
Interactive Visualizations and Real-Time Data Integration: In some embodiments, the framework includes interactive visualizations that allow users to explore different scenarios and their potential impacts on clinical effectiveness. Additionally, the model integrates newly acquired or up-to-date data inputs to continuously update the decision-making framework, ensuring that the analysis remains relevant and accurate. Additionally, all aspects may be substituted for forecasted model parameters, anticipated model structures, and forecasted data, enabling a strategic analysis of decision-options that supports decision-making for robust performance. Overall, certain aspects relating to certain embodiments provide a unique and useful framework for evaluating medical technology strategies, making it a novel contribution to the field.

Integration of health-related outcomes. Certain embodiments combine MATE and CEA models into a single decision support framework, and thus may be extended to holistically include other measures of health-related value. For example, health-related outcomes such as Equity, Value of Hope, and Reduction in Uncertainty, to name a few (see ISPOR “value flower”), are also considered in development, reimbursement, and investment decisions, and are thus important metrics in strategic decision-making. It is known that strategic decision-makers may aggregate multiple measures of value, perhaps using weighted sum approaches like multi-criteria decision analysis (MCDA). In some aspects of some embodiments, this approach allows side-by-side comparison of strategies in terms of their performance across a multitude of health-related outcomes (inclusive to clinical effectiveness), including aggregate indicators of health-related “value”, which is not done in existing practices. Any relationships between health-related value and development decision-options can therefore be explored and included in a holistic strategic analysis that is novel and useful.

According to one aspect a computer-implemented method is provided comprising modeling, in one or more computer systems, a first model representing a plurality of decision-options as treatment options in one or more clinical decision situations, modeling, in one or more computer systems, a second model evaluating the plurality of treatment options in terms of one or more health-related outcomes, and providing an output indicating one or more health-related outcomes associated with one or more of the decision-options. According to some embodiments, the act of representing the plurality of decision-options as treatment options includes enumerating possible system design decision-options and/or feasible combinations of design decision-options.

According to some embodiments, the act of enumerating system design decision-options and/or feasible combinations of decision-options includes at least one of a group comprising generating possible system design decision-options, refining possible system design decision-options, and listing possible system design decision-options. According to some embodiments, the design decision-options are system architectural decision-options that conceptually constrain a set of design decision-options, including design decision-options that are mutually exclusive and collectively exhaustive (MECE).

According to some embodiments, the acts of modeling include modeling sensitivity and/or uncertainty using a range of possible and/or imagined representations and/or evaluations for the plurality of decision-options, including representing and evaluating one or more of the decision-options using at least one of a group comprising maximum and minimum values, probability distributions; and lookup tables. According to some embodiments, the act of providing an output includes analyzing at least one of sensitivity and uncertainty, including by indicating a range of at least one of possible and predicted health-related outcomes, including at least one of the following acts of indicating for one or more of the decision-options indicating maximum and minimum values, indicating probability distributions, indicating histogram, cumulative distribution, and cumulative probabilities of individual values, and indicating ranges of values for the plurality of outcomes.

According to some embodiments, the act of indicating a range of at least one of possible and predicted health-related outcomes includes providing an output indicating summary and prospective information about the act of analyzing the at least one of sensitivity and uncertainty, including indicating key sources of uncertainty and hypothetical outputs under simulated changes in uncertainty, by reducing uncertainty with more data. According to some embodiments, the prospective information includes at least one simulated scenario.

According to some embodiments, the act of providing an output indicating summary and prospective information about the sensitivity and/or uncertainty analysis includes indicating the conditions under which one or more decision-options may be selected. According to one embodiment, the conditions under which the one or more decision-options may be selected includes at least one of Pareto dominance, cost-effectiveness, willingness-to-pay, and overall conditions for reimbursement, over one or more alternative decision-options.

According to one embodiment, the acts of modeling and evaluating include determining a maximum reasonably achievable value for one or more health-related outcomes and establishing an exhaustive list of technology development and clinical development decision-options. According to some embodiments, the act of determining a maximum reasonably achievable value for one or more health-related outcomes includes using a range of possible and/or imagined representations to determine an optimal treatment, including representing and evaluating one or more of the decision-options using at least one of a group comprising maximum and minimum values, probability distributions, and lookup tables.

According to some embodiments, the act of providing an output includes simultaneously indicating the optimal treatment on one or more dimensions of an evaluation along with a set of decision-options. According to some embodiments, the act of modeling the plurality of decision-options includes decision-options that related to both system design and clinical development decision-options. According to some embodiments, both system design and clinical development decision-options include at least one of a group comprising target indications and clinical trial design.

According to some embodiments, the act of providing an output includes visualizing tradeoffs among two or more of the evaluated plurality of decision-options, including visually indicating one or more of a group comprising health-related outcomes and related measures of healthcare value simultaneously with another measure of performance and/or system attribute. According to some embodiments, another measure of performance and/or system attribute includes at least one of a group comprising development time, risk, production cost, energy usage, physical envelope, and technology readiness level. According to one embodiment, wherein the visualizing is performed using a tradespace plot.

According to some embodiments, the act of providing an output includes indicating a measure of clinical effectiveness associated with one or more of the decision-options, including indicating clinical effectiveness as a measure of impact on quality of life and/or duration, such as QALYs, DALYs, evLYGs, and/or similarly derived metrics. According to some embodiments, the act of providing an output includes indicating derivative metrics of healthcare value stemming from clinical effectiveness modeling, for example when implemented as a decision tree and/or Markov model, including derivative metrics like incremental cost-effectiveness ratio (ICER).

According to some embodiments, the act of providing an output includes indicating metrics stemming from derivative analyses and visualization techniques related to clinical effectiveness modeling, for example measures emerging from a willingness-to-pay (WTP) analysis. According to some embodiments, the act of providing an output that indicates one or more health-related outcomes includes indicating outcomes emerging from healthcare value assessment frameworks. According to some embodiments, the healthcare value assessment frameworks include at least one or more of a group comprising clinical effectiveness, net costs, productivity, family spillovers, reduction in uncertainty, insurance value, fear of contagion and disease; severity of disease, value of hope, real option value, equity, and scientific spillovers.

According to some embodiments, the method includes weighting a plurality of indicators of overall value of the plurality of healthcare value assessment frameworks that incorporate stakeholder preferences. According to one embodiment, the act of indicating outcomes emerging from healthcare value assessment frameworks includes visually indicating conditional status. According to some embodiments, the act of indicating includes indicating whether a decision-option is reimbursable according to a user-defined threshold and includes indicating an uncertainty thereof.

According to some embodiments, the act of providing an output that indicates one or more health-related outcomes includes indicating patient-reported outcome(s) (PROs) associated with one or more of the decision-options, including indicating patient preferences and/or aggregate measures of PROs, for example with multi-criteria decision analysis (MCDA). According to some embodiments, the act of indicating the patient-reported outcome(s) (PROs) associated with one or more of the decision-options includes indicating effects of uncertainty in patient preferences.

According to some embodiments, visualizing tradeoffs among two or more of the evaluated plurality of decision-options includes simultaneously displaying a plurality health-related outcomes, including known and/or imagined aggregate values. According to some embodiments, the known and/or imagined aggregate values include metrics reported as a weighted sum including multi-criteria decision analysis (MCDA).

According to one aspect, a computer-implemented method is provided. The method comprises modeling, in a computer system, a first model representing a plurality of decision-options, modeling, in the computer system, a second model representing an effectiveness of the plurality of decision-options, and providing an output indicating performance of a set of design options selected from the plurality of decision-options against a plurality of metrics. According to some embodiments, the plurality of decision-options relates to at least one treatment of a patient, and wherein the act of representing, by the second model, the effectiveness of the plurality of decision-options includes an act of measuring a clinical effectiveness of at least one treatment of the patient. According to some embodiments, the method further comprises an act of performing a cost-effectiveness analysis to determine a most cost-effective treatment option.

According to some embodiments, the act of performing the cost-effectiveness analysis includes calculating an aggregate measure of clinical effectiveness including measures of quality of life and years of survival (e.g., quality-adjusted life years (QALYs), disability adjusted life years (DALYs), and equivalent value of life-years gained (evLYGs)), for each treatment option. According to some embodiments, the act of performing the cost-effectiveness analysis further includes performing a probabilistic analysis to account for uncertainty in outcomes of each treatment option. According to some embodiments, the act of performing the probabilistic analysis includes performing Monte Carlo simulations.

According to some embodiments, the act of providing an output includes generating a visualization of the set of design options in terms of one or more attributes and/or dimensions of value, including with a visualization of uncertainty. According to some embodiments, the act of providing an output includes generating one or more visualizations of different combinations of design options. According to one embodiment, the visualization includes interactive elements that allow users to explore different scenarios and their potential impacts on clinical effectiveness.

According to one embodiment, the act of providing an output further includes generating a report summarizing a recommended set of design options and their associated clinical effectiveness metrics. According to one embodiment, the method further includes integrating real-time data inputs to continuously update a decision-making framework. According to one embodiment, the method further includes prioritizing decision-options based on user-defined criteria.

According to one aspect, an apparatus for supporting decision-making in medical technology strategy is provided. The system comprises a Multi-Attribute Tradespace Exploration (MATE) module configured to generate system architectural decision-options and plot them in a tradespace plot, a Clinical Cost-Effectiveness Analysis (CEA) module configured to calculate clinical effectiveness metrics, including measures of quality of life and years of survival (e.g., quality-adjusted life years (QALYs), disability adjusted life years (DALYs), and equivalent value of life-years gained (evLYGs)), for the decision-options (including combinations of decision-options), a relationship positing module configured to establish relationships between the decision-options and their impact on clinical effectiveness, a probabilistic analysis module configured to perform Monte Carlo simulations to account for uncertainty in performance of the decision-options, a visualization module configured to provide a coordinated visualization of the decision-options in terms of multiple dimensions of value, their uncertainty, and attributes of other treatment options, wherein the apparatus integrates outputs of the MATE module and the CEA module to generate a combined model that supports decision-making in medical technology strategy.

According to some embodiments, the MATE module is further configured to evaluate trade-offs between different architectural decision-options based on multiple criteria including cost, performance, and risk. According to one embodiment, the multiple criteria includes one or more measures of utility including multi-attribute utilities. According to some embodiments, the CEA module is further configured to incorporate patient-reported outcomes in a calculation of at least one of a group comprising clinical effectiveness metrics, other health-related metrics, and multi-criteria measures of treatment performance or value.

According to one embodiment, the relationship positing module is further configured to use one or more machine learning algorithms to establish relationships between a function of a technology and its impact on clinical effectiveness. According to one embodiment, the function of the technology includes one or more of the decision-options that are provided as inputs to the one or more machine learning algorithms. According to one embodiment, the probabilistic analysis module is further configured to perform sensitivity analysis to identify one or more most influential variables affecting outcomes of the decision-options.

According to one embodiment, the probabilistic analysis module is further configured to perform one-way sensitivity analysis on a group of variables to identify one or more influential variables. According to one embodiment, the probabilistic analysis module is further configured to replace one or more influential variables with probabilistic distributions. According to one embodiment, the probabilistic analysis module is further configured to perform probabilistic sensitivity analysis.

According to one embodiment, the probabilistic analysis module is further configured to output, to a display, probabilistic performance relative to other decision-options or existing treatments in a standard of care. According to some embodiments, the visualization module is further configured to generate interactive visualizations that allow users to explore different scenarios and their potential impacts on clinical effectiveness. According to one embodiment, the combined model integrates real-time data inputs to continuously update a decision-making support framework.

According to one embodiment, the apparatus is further configured to generate a report summarizing a set of design options and their associated clinical effectiveness metrics. According to one embodiment, the associated clinical effectiveness metrics include clinical effectiveness relative to a reference treatment, for example, a dominant treatment in a standard of care or anticipated competitive situation. According to one embodiment, the MATE module includes a feature for prioritizing decision-options based on user-defined criteria.

According to one aspect, a non-transitory computer-readable medium storing instructions that, when executed by a processor, cause the processor to perform a method is provided. The method comprises modeling, in a computer system, a first model representing a plurality of decision-options, the plurality of decision-options including an associated cost, modeling, in the computer system, a second model representing an effectiveness of the plurality of decision-options, optimizing, by the computer system, the plurality of decision-options of the first model and the effectiveness of the plurality of decision-options of the second model, and providing an output indicating performance of a set of design options selected from the plurality of decision-options against a plurality of metrics.

Still other aspects, examples, and advantages of these exemplary aspects and examples, are discussed in detail below. Moreover, it is to be understood that both the foregoing information and the following detailed description are merely illustrative examples of various aspects and examples and are intended to provide an overview or framework for understanding the nature and character of the claimed aspects and examples. Any example disclosed herein may be combined with any other example in any manner consistent with at least one of the objects, aims, and needs disclosed herein, and references to “an example,” “some examples,” “an alternate example,” “various examples,” “one example,” “at least one example,” “this and other examples” or the like are not necessarily mutually exclusive and are intended to indicate that a particular feature, structure, or characteristic described in connection with the example may be included in at least one example. The appearances of such terms herein are not necessarily all referring to the same example.

### BRIEF DESCRIPTION OF THE DRAWINGS

Various aspects and embodiments will be described with reference to the following figures. It should be appreciated that the figures are not necessarily drawn to scale. Items appearing in multiple figures are indicated by the same or a similar reference number in all the figures in which they appear. The figures are included to provide illustration and a further understanding of the various aspects and embodiments and are incorporated in and constitute a part of this specification but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 illustrates how Extended Cost-Effectiveness Analysis (ECEA) is a useful methodology which policy analysts use to explore trade-offs in health and non-health criteria.

FIG. 2 depicts the concept of a tradespace plot used in Multi-Attribute Tradespace Exploration (MATE), illustrating the relationship between cost and value for various decision-options.

FIG. 3 illustrates a schematic decision tree for a simple CEA model for epilepsy (e.g., drawn using an industry-standard CEA modeling tool called TreeAge).

FIGS. 4A-4B illustrate a decision tree and corresponding table of results for a CEA model comparing a new treatment (i.e., a rectal device), a hydrogel spacer, and no therapy options for proctitis treatment.

FIG. 5 shows an example system and architecture according to various embodiments.

FIG. 6 shows an example process for determining a recommendation set of design options according to various embodiments.

FIG. 7 shows an example detailed block diagram of a system and architecture according to various embodiments.

FIG. 8 shows an example process for analyzing system design options in a clinical design system according to various embodiments.

The figures depict various embodiments of the present invention for purposes of illustration only. One skilled in the art will readily recognize from the following description that other alternative embodiments of the structures and methods illustrated herein may be employed without departing from the principles of the invention described herein.

### DETAILED DESCRIPTION

Some embodiments relate to the field of healthcare for humans and/or animals, and methods for optimizing such care. Clinical cost-effectiveness analysis is a modeling practice used to describe and explore alternatives in the standard of care, as well as a practice that payers use to determine whether to reimburse a treatment option. It is therefore critically important in most modern economies when considering business strategy. In some embodiments, it is appreciated that it may be helpful to combine clinical cost effectiveness (CEA) and muti-attribute tradespace exploration (MATE) modeling related to new treatment and diagnostic options to pursue insights into how strategic decisions may impact clinical and reimbursement potential.

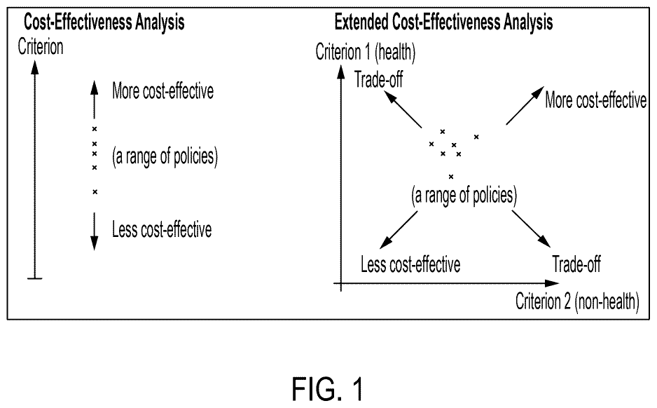

FIG. 1 illustrates a comparison between Cost-Effectiveness Analysis (CEA) and Extended Cost-Effectiveness Analysis (ECEA) using a tradespace plot. The left side of the figure shows a single criterion for cost-effectiveness, with policies plotted along a vertical axis indicating more or less cost-effective options. The right side of the figure shows an extended analysis with two criteria: Criterion 1 (health) and Criterion 2 (non-health). The plot indicates trade-offs between these criteria, with policies plotted to show their relative cost-effectiveness in both dimensions. In FIG. 1, each “x” on the X-Y plot (right) is a policy. Its position on the plot indicates its performance against two criteria, which allows the reader to see trade-offs in policies. It is also possible to combine criteria using weighting into a multi-attribute utility.

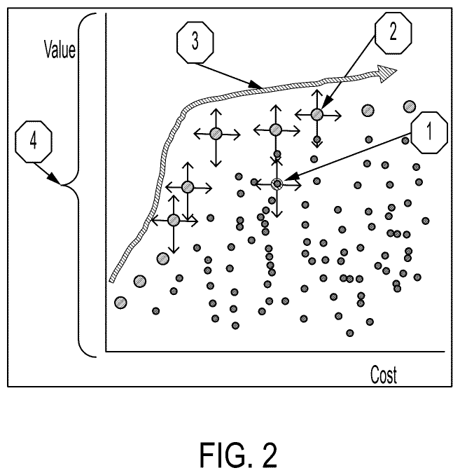

FIG. 2 depicts the concept of a tradespace plot used in Multi-Attribute Tradespace Exploration, illustrating the relationship between cost and value for various decision-options. The plot shows multiple decision-options represented by dots, with arrows indicating the uncertainty in their value and cost. The shaded line represents the Pareto frontier, indicating the most efficient trade-offs between cost and value. In addition to comparing “Value” to “Cost” (e.g., in FIG. 2), such a relation may be used to compare all sorts of tradeoffs. For example, an individual could compare system size to cost. Further, a person could also normalize “Value” as a “Utility” score (from 0 to 1) and even combine multiple single-attribute utility metrics into a multi-attribute utility metric.

In some embodiments, it is appreciated that Clinical Cost-Effectiveness Analysis (CEA) is a modeling practice that may be used to provide indicators of “Value” in relation to healthcare. For example, a healthcare payor may evaluate the clinical performance of a treatment relative to a reference treatment and determine whether the reimbursement is warranted by the expected improvement in clinical performance, as applicable. An example indicator of “Value” from CEA that may replace “Value” in the tradespace plot is the Quality-Adjusted Life Year (QALY). Additionally, variants on the QALY may be used, such as the Equal Value of Life Years Gained (evLYG), the Disability-Adjusted Life Years (DALY), or merely the extension of life years. Other measures of health-related outcomes may be used as indicators of “Value”, such as those represented in the ISPOR ‘Value Flower’, which includes Equity, Value of Hope, and Reduction in Uncertainty, to name a few. Patient-Reported Outcomes (PROs) may also indicate “Value”. “Value” indicators may be combined into multi-criteria (e.g., weighted average) metrics. Such indicators of “Value” may combined with other health-related value analyses, including a Willingness-To-Pay (WTP) analysis or an evaluation of Incremental Cost-Effectiveness Ratio (ICER), for example.

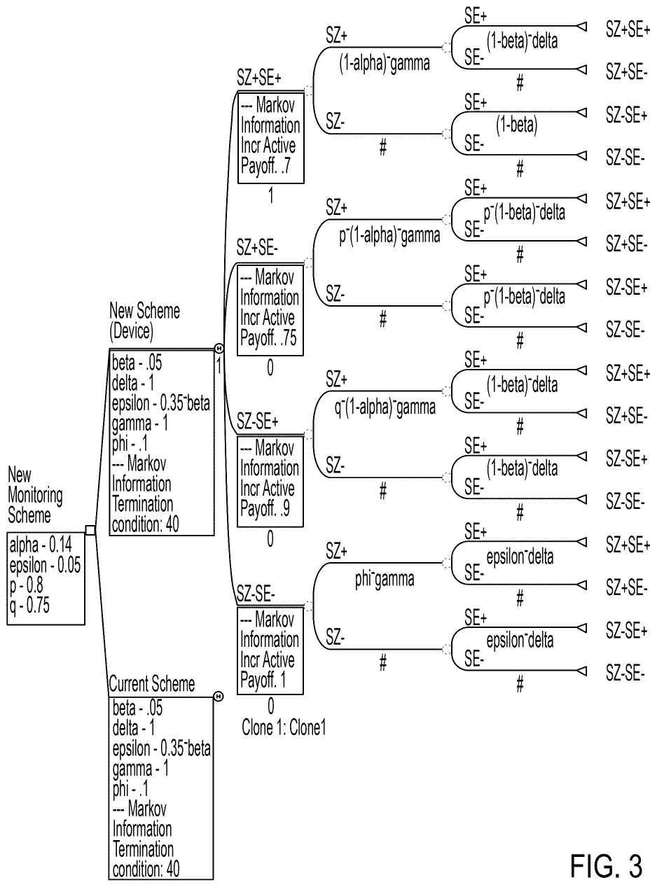

FIG. 3 illustrates a schematic decision tree for a simple CEA model for epilepsy built using an industry-standard tool called TreeAge, though decision trees and Markov models can be built using programming languages, spreadsheet software, etc. Thedecision tree shows two main branches established as Markov models: one for the “Current Scheme” and one for the “New Scheme”, the details (branches) for which are not visible (collapsed). The “Current Scheme” assumptions come from research data. The “New Scheme” assumptions can be developed from a variety of factors, including expert input, preliminary data, comparative studies, hypothetical assumptions to be challenged, and other assumptions or heuristics. These assumptions depend on which technology decision is being explored. Each branch includes chance nodes with outcomes and their associated probabilities, leading to a distribution of payoffs (often calculated as QALYs, for example).

Though simple decision trees can be used to calculate a single expected value, it is known that CEA modelers conduct probabilistic analyses, and according to some embodiments, a probabilistic analysis is conducted where constants are replaced with distributions of variables, often defined by probability distribution functions. If, for example, one executes the analysis 1000 times with random number generators (e.g., by the computer system), a distribution of outcomes (in QALYs) is obtained. In some embodiments, this distribution in outcomes is provided as an input into the tradespace analysis.

Though CEA modeling can involve a variety of decision tree designs from simple to complex, and the simulation and statistical practices can range from simple to sophisticated, this example can give an idea of the unique practices of CEA modeling and how a system that incorporates CEA models into MATE models is particularly non-obvious, and opens the door to include a multitude of measures of value (e.g. Equity, Value of Hope, Reduction in Uncertainty, etc.).

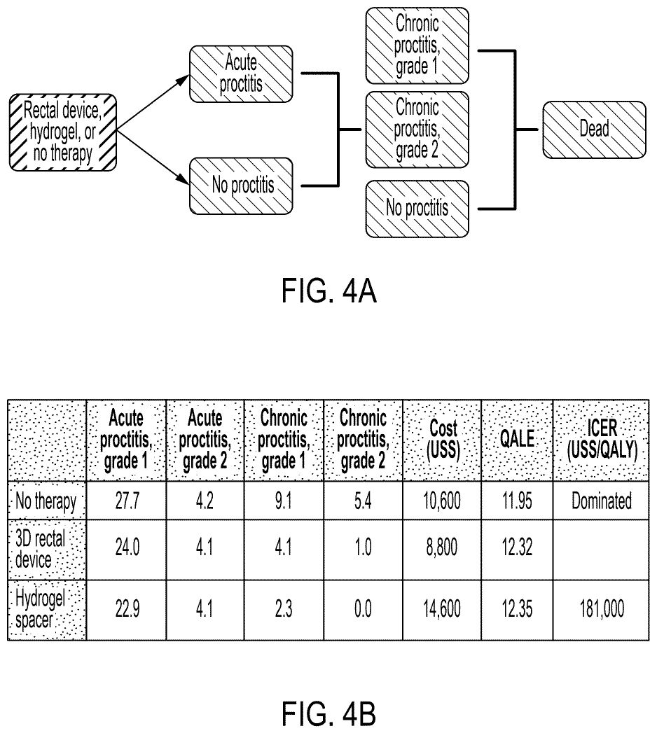

FIGS. 4A-4B originate from a recently published research paper (Byrne et al., 2021) and illustrates how laboratory researchers interested in the commercialization potential of a new medical technology used CEA to compare the anticipated health-related performance (see results in corresponding table) rectal device, hydrogel, and no therapy options for proctitis treatment. The decision tree shows the possible outcomes for each treatment option, including acute and chronic proctitis of different grades and death. The table provides the cost, quality-adjusted life expectancy (QALE), and ICER for each treatment option. To extend this type of analysis to incorporate the practices of Multi-Attribute Tradespace Exploration (MATE) is novel and useful, for example by making assumptions explicit in a way that enhances explainability of results.

### Example System Implementation

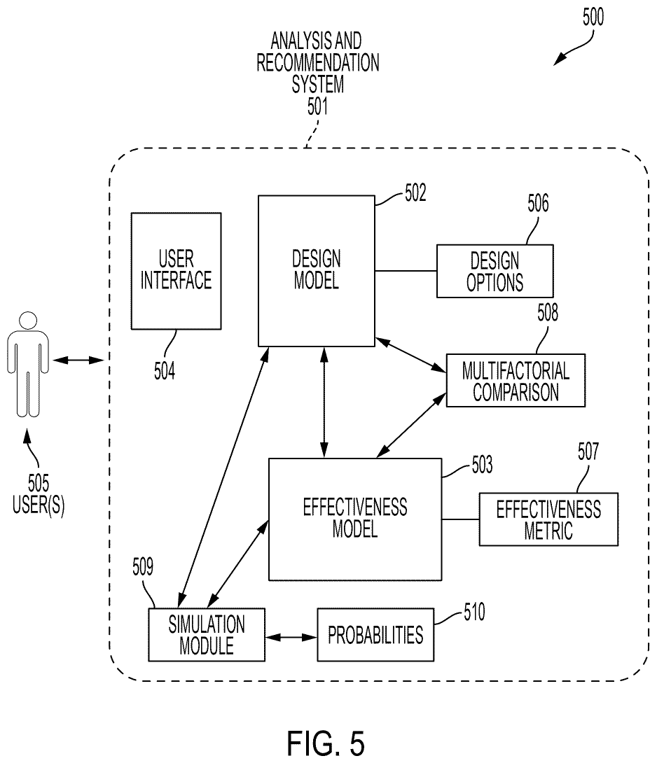

FIG. 5 shows one example of a system architecture capable of implementing various aspects of the present invention. In particular, FIG. 5 shows an analysis and recommendation system 501 which is capable of interacting with one or more users (e.g., user(s) 505) to provide one or more insights to the user. For example, and as discussed further below, the system may model certain aspects of the design and allow the user to view and model aspects of the system, such as dominant and dominated solutions, trade-offs with other metrics like cost, quality, risk, development time, etc., main sources of uncertainty and relative threats to concepts, among other insights. System 501 may operate as part of a distributed computing system 500 which may include one or more end systems, services, and/or computer networks. System 501 includes one or more components including a user interface 504 which interacts with a user to determine one or more design options 506 that can be expressed within a design model 502. Further, system 501 permits the creation of a clinical effectiveness model 503 which relates clinical effectiveness (and related metrics) 507 to one or more design options 506.

System 501 further includes a multifactorial comparison component 508 which allows different permutations of multiple design options and their effects to be modeled and optimized. System 501 also includes a simulation module 509 that uses probabilities 510 to simulate different options and their probabilistic results. In some implementations, Monte Carlo simulations may be used to leverage probabilistic analysis to account for uncertainty in particular outcomes which enhances the decision-making process.

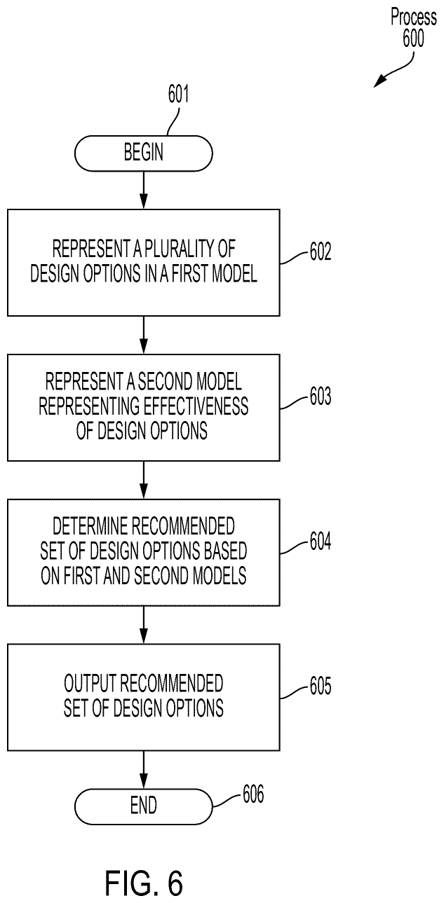

FIG. 6 shows a process 600 for determining a recommendation set of design options according to various embodiments. At block 601, process 600 begins. At block 602, using a computer system, user or other entity represents a plurality of design options in a first model. For example, the user may operate a user interface to define design options and characteristics of a system to be designed within the interface. Such a set of design options may be stored within a memory of the computer system and manipulated through one or more executable programs.

At block 603, the system is used to represent a second model which represents and clinical effectiveness of each of the design options. Once defined, the system may determine a recommended set of design options based upon the first and second models at block 604. The system may perform one or more analysis that identifies and visualizes clinical effectiveness of design options which may be then presented to the user or other system (e.g., within a user interface, presented as a graph, summary or other report format). In some embodiments, results may be displayed to the user within a user interface such as a graph or other representation. At block 606, process 600 ends.

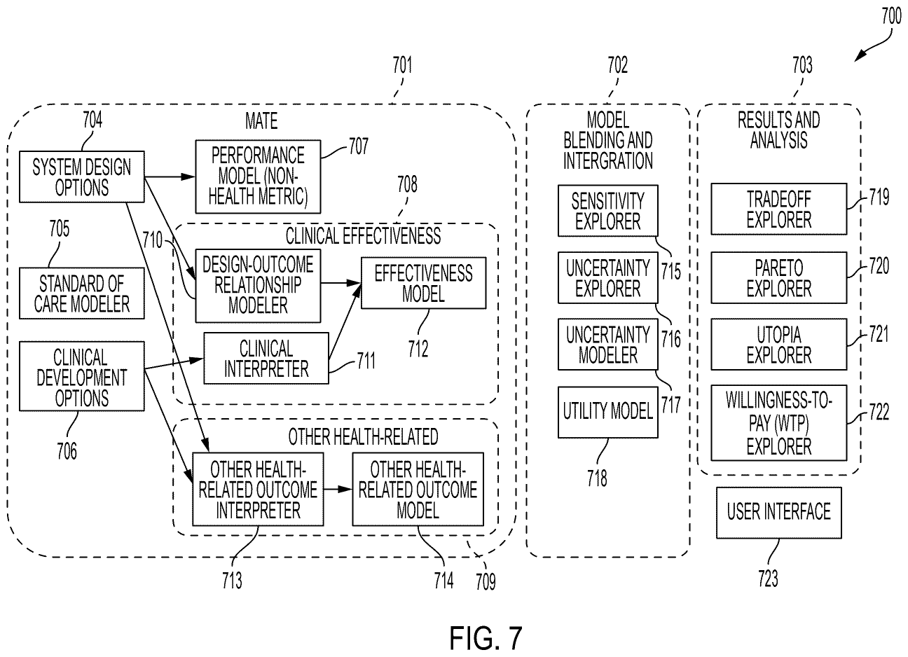

FIG. 7 shows an example detailed block diagram of a system and architecture 700 according to various embodiments that may be used by one or more users to interactively model and select design options that can contribute toward a solution (e.g., a treatment, combination of treatments, or coordination with value-enhancement options like drug delivery, diagnosis, and digital health products, for example). The system and architecture 700 include one or more modules that interact to represent system designs and models used for evaluation of those designs. In particular, the system 700 includes a MATE system 701 which further includes system design options 704 that represent various architectural decisions that can be made in the development of medical technology and its clinical enablement. These options may include different configurations, features, and functionalities that can be incorporated into the system, or accompany the system directly or indirectly. The system design options 704 serve as the input for the Multi-Attribute Tradespace Exploration (MATE) 701, which models the performance of these options against any number of criteria, including cost, performance, measures of stakeholder utility, clinical performance, and development risk, to name a few. Because of the strategic nature of the evaluations, the system design options 704 are represented as mutually exclusive and collectively exhaustive (MECE) decision-options that conceptually constrain the universe of decision-options in the analysis, which allows for a holistic and exhaustive evaluation of all available options.

The standard of care modeler 705 characterizes the current standard of care for one or more medical conditions in terms of patient populations, their journeys, decision points, available treatments, trends in related technology, and potential disruptions. The standard of care modeler 705 scopes and defines the models of health-related outcomes (e.g., in Cost-Effectiveness Analysis (CEA) models) within which clinical development options 706 can be evaluated in the MATE 701. The standard of care modeler 705 includes data on treatments (and combinations of treatments), their effectiveness, and associated costs, establishing a baseline from which to understand the potential impact of new technologies on patient outcomes and healthcare costs. The clinical development options 706 represent various strategies for establishing evidence for medical technology prescription, adoption, and reimbursement, including different clinical trial scoping options, evidence gathering strategies, regulatory pathways, and commercialization strategies. The clinical development options 706 are evaluated within the MATE 701 framework to determine their potential impact on clinical effectiveness (and related metrics) and other health-related outcomes.

The performance model (non-health metric) module 707 quantifies the performance of the system design options 704 based on non-health-related metrics (e.g., cost, size, weight, reliability, etc.). All modeling assumptions are captured and codified, such as the structure, parameters, and impacts to assumptions for each design option. As with the clinical effectiveness 708 and other health-related 709 modules, the performance model (non-health metric) 707 is flexible and adaptive to interactions with the sensitivity explorer 715, the uncertainty explorer 715, the uncertainty modeler 716, and the user interface 723. For example, the uncertainty modeler 716 facilitates replacement of deterministic variables with probability distributions.

The clinical effectiveness model 708 establishes models and engineered data sets that evaluate system design options 704 in terms of patient outcomes. This model uses clinical data and assumptions from a multitude of sources (e.g., user input, lit review, clinical trial read-outs, engineered data sets, drug protocols, etc.) to estimate the effectiveness associated with the different decision options, in terms of quality-adjusted life years (QALYs), for example. The clinical effectiveness module 708 is integrated with the MATE 707 framework to provide a holistic evaluation of the trade-offs between different design options.

Note that “effectiveness” can be any measure of “value” in the standard of care 705, as well as aggregate measures (e.g., weighted sum), engineered data sets (e.g., complex read-outs), and predictive outputs (e.g., through Bayesian methods). The user may interact with any of the system elements through the user interface 723 to formulate a model that is as simple or as complex as one desires, for example by extending the standard of care modeler 705 to include any measure(s) of effectiveness, known, anticipated, predicted, hypothesized, or hypothetical.

The design-outcome relationship modeler 710 establishes relationships between the system design options 704 and their impact on the clinical effectiveness model 712. This model interprets system design options 704 based on the learnings of the standard of care modeler 705, which involves using any combination of user input, machine learning, and generative artificial intelligence to connect system design options 704 to their impacts on the effectiveness model 712. In an iterative capacity, these impacts are adaptive to downstream interactions, for example with the sensitivity explorer 715, uncertainty explorer 716, uncertainty modeler 717, and inputs from the user interface 723.

The clinical interpreter 711 establishes relationships, parametric constraints, variables, and uncertainty modeling constraints based on the clinical development options 706, to be codified into the clinical effectiveness model 712. The clinical interpreter 711 provides a summary of the potential impact of different clinical development options 706 on patient outcomes, helping decision-makers to prioritize the most relevant options in the analysis and inform strategic decision-making.

The effectiveness model 712 organizes and reports clinical cost-effectiveness and related measures of clinical outcomes, with uncertainty as guided by the uncertainty modeler 717, incorporating discrete measures like additional survival years, freedom from disability, freedom from pain, and other indicators of health and wellness, as may be reported by clinicians and/or patients. Derivative metrics are also established, such as ICER and inputs to and results from a WTP analysis.

Inclusion of other health-related 709 measures of performance in the MATE 701 model requires first another health-related outcome interpreter 713 to translate system design and clinical development options into their impacts on other health-related outcomes (i.e., those aside from clinical effectiveness and its related and derivative metrics). Additionally, further modeling may be required, such as macroeconomic modeling, for example, which is handled in the other health-related outcome model 714. The other health-related 709 module ultimately organizes and reports other health-related outcomes as a function of system design and clinical development options, with uncertainty as guided by uncertainty modeler 717.

The other health-related outcome model 714 evaluates the impact of the system design options 704 on other health-related outcomes such as Equity, Value of Hope, etc., and may be extended to include patient-reported outcomes, models of patient preferences, and adherence, for example. As applicable, weighted sum and other derivative measures of health-related outcomes are made available.

The model blending and integration component 702 evaluates the combined models and resulting data sets from the clinical effectiveness model 708, the performance model (non-health metric) 708, and the other health-related outcome model 709. Feedback can be provided into the MATE 701 module to account for sensitivities, uncertainties, ambiguities, and unknowns, so that the model can be enhanced for results and analysis 703.

The sensitivity explorer 715 performs sensitivity analysis to identify the influential variables affecting the outcomes of the decision-options. This component helps to identify the assumptions and sources of uncertainty that can be challenged and/or removed, allowing decision-makers to prioritize the promising options. The sensitivity explorer 715 establishes high and low values (guided by user input), outputs tornado plot data, and identifies the most sensitive variables to consider converting static variables to probabilistic distributions. The result is guided by inputs from the user interface 723.

The uncertainty explorer 716 evaluates the impact of uncertainty on the effects of the decision-options (704 & 706) in the MATE 701 model. This component uses probabilistic analysis and Monte Carlo simulations to account for uncertainty in the outcomes, providing a comprehensive assessment of the potential impact of different design options. In some implementations, uncertainty explorer 716 combines disparate data sets and user input to establish probabilistic distributions of data to replace deterministic variables, as applicable. Other sources of uncertainty not related to decision-options, such as uncertain sources of health-related benefits from the standard of care modeler 705 or uncertainty associated with clinical development options 706, for example, can be identified and blended into the model as well.

The uncertainty modeler 717 performs probabilistic analysis to account for uncertainty in the effects of the system design options 704 and clinical development options 706. This component uses Monte Carlo simulations to generate a distribution of outcomes, providing a comprehensive assessment of the potential impact of different design options. A model validation process runs sensitivity study and probabilistic sensitivity study using distributions from the uncertainty modeler 717 to validate that the model runs correctly for all possible values, and helps ensure that the model outputs are reasonable.

The utility model 718 interprets outputs from the performance model 707, the effectiveness model 712, and the model of other health-related outcomes 712, using other parameters that capture relationships between these outputs, to produce measures of stakeholder utility (or multi-attribute utility). The results and analysis component 703 provides a comprehensive summary of the potential impact of different design options on performance, clinical effectiveness (and related metrics), and other health-related outcomes. This component includes visualizations and reports that help decision-makers to make robust decisions.

For example, the tradeoff explorer 719 provides a coordinated visualization of the trade-offs between different combinations of design and clinical development options in terms of multiple attributes and dimensions of value, and their associated uncertainty. This component includes interactive elements that allow users to explore different scenarios (e.g., combinations of strategies, different stakeholder or user preferences, different multi-attribute weightings, current vs. forecasted assumptions, deterministic vs. probabilistic assumptions, etc.) and their potential impacts on performance, effectiveness, other health-related outcomes, and measures of stakeholder utility.

The utopia explorer 721 establishes “favorability” for each metric (e.g., lower cost is more favorable) and identifies the idealized performance (i.e., “utopia”). It is known that the favorable extrema for each metric can represent utopia (e.g., $0 cost and stakeholder utility=1). It is uncommon and perhaps not known that utopia is represented as an explicit, theoretical maximum, though theorized maximum performance is common within technology roadmapping. It is novel, however, to establish a maximum possible health-related related outcome and include this “Utopia Treatment” as a probabilistic “utopia point” (common MATE terminology) in the tradespace plot. Including this artifact in the analysis can help decision-makers understand the maximum achievable overall performance, which can help anticipate the potential for investment(s) and understand the potential for threats in the standard of care, for example.

The Pareto explorer 720 identifies the Pareto frontier of dominant solutions which could be known as the set of solutions for which (according to deterministic calculations) no other solution dominates without an accompanying decrease in favorability against the other metric shown in the tradespace plot.

The willingness-to-pay (WTP) explorer 722 explores derivate metrics and analysis of the CEA. This component tailors a WTP analysis report for the results and the user inputs. Incremental cost-effectiveness ratios (ICERs) are calculated, too, providing a comprehensive assessment of the potential impact of different design options.

The user interface 723 provides a coordinated visualization of the development and investment decision-options in terms of multiple dimensions of value, their uncertainty, and other attributes of interest. This component includes interactive elements that allow users to holistically explore different scenarios and their potential impacts on important strategic metrics like clinical effectiveness. User interface 723 also interacts with user to evaluate performance of the designed system given different assumptions, as well as relative performance of more than one system design alternative, tradeoffs in system performance against multiple metrics, and the impact of uncertainty on anticipated system performance, comparing performance, exploring tradeoffs, and understanding the role of uncertainties.

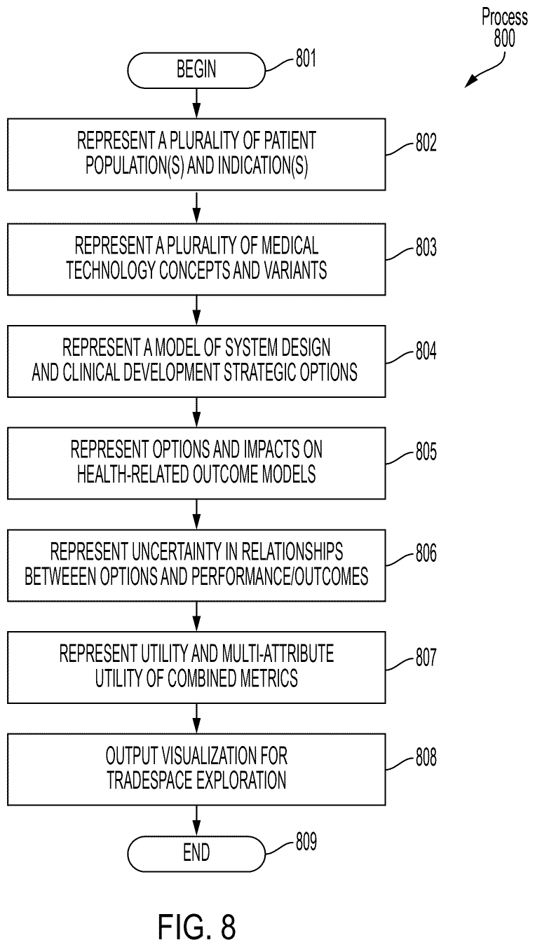

FIG. 8 shows an example process for analyzing system design options in a clinical design system (e.g., system 700 of FIG. 7) according to various embodiments. In particular, FIG. 8 illustrates a flowchart of a process 800 for supporting decision-making in medical technology strategy. The process begins at step 801 and proceeds through several stages to represent and analyze various aspects of medical technology concepts and their impacts on health-related outcomes.

At block 801, process 800 begins. At block 802, a user and/or system represents a plurality of patient population(s) and indication(s). This step involves identifying and representing different patient populations and medical indications that are relevant to the analysis and storing engineered data sets that represent these patient populations and medical indications in a memory of a computer system. Further, this step may include defining a representation of a roadmap of the standard of care (past, present, forecast, and potential disruptions) related to the medical strategy. This step may also include establishing one or more health-related outcome models (e.g., CEA model, including any number of parameters and structural assumptions) which captures and interprets clinical data, physician input, and clinical modeling expertise.

At block 803, a plurality of medical technology concepts and variants is represented. This step involves identifying and representing various medical technology concepts and their possible variants that make up the standard of care. Technologies, their variants, and alternative treatments may be defined according to best practices in MATE modeling, and may fully enumerate the feasible set of options and their combinations. At block 804, a model of system design and clinical development strategic options is created and stored. This step involves creating a model that includes different system design options and clinical development strategies, including defining which combinations are feasible. This step establishes the bedrock of the MATE model. At block 805, options and their impacts on health-related outcome models are posited. This step involves modeling the potential impacts of different options on health-related outcomes.

At block 806, the system is used to represent uncertainty in relationships between options and performance & outcomes. This step involves accounting for uncertainty in the relationships between different options and their performance & outcomes. This may include identifying one or more significant variables through sensitivity study (e.g., via a tornado plot), representing sensitive variables as distributions of possibilities, and validating model behavior. At block 807, utility and multi-attribute utility of combined metrics are represented. This step involves representing the utility and multi-attribute utility of various combined metrics to evaluate different options against overall measures of stakeholder value.

At block 808, the system outputs one or more visualizations for tradespace exploration to a user or other computing entity. This step involves generating visualizations that allow for the exploration of the tradespace, showing trade-offs in any combination of metrics across different options. The system may be configured to identify a Pareto frontier which identifies the set of undominated solutions. Uncertainties are plotted, as applicable, in a way that supports strategic decision-making. The user may explore trade-offs between competing objectives and consider uncertainty to identify robust solutions. These steps may also involve identifying a “utopia” point in the analysis which identifies the maximum theoretically achievable outcome against each metric, which may include indicating uncertainty on the plot. At block 809, process 800 ends.

### Example Implementation

In some implementations, the approach combines MATE and CEA models to support system architecture of a system that enhances the value of an existing (or emerging) treatment, such as a drug delivery system. Starting with a MATE model, the system would model the architectural decision-options (e.g., as shown in FIG. 4B which shows a table of combinable options for a drug delivery system). Then, the system would plot the performance of all feasible combinations of options in the tradespace plot (X-Y plot). This modeling would be followed by modeling a CEA.

Next, the system would be used to define outcomes that the drug delivery pump can impact in terms of QALYs. For example:
A computer user (e.g., a user performing operations on a computer interface) could hypothesize (with uncertainty) that an on-body wearable pump increases quality of life by getting patients out of the house, thereby increasing health and lifespan.
The user could also anticipate that a peristaltic pump preserves the integrity of the drug (with some uncertainty) and prevents users from infusing too quickly, for example, thereby increasing the effectiveness of the drug and reducing side effects.
A user could further anticipate that control with a mobile phone makes patients feel less like patients and gives them hope for living more normal lives, thereby affecting other health-related outcomes like Value of Hope.

So, what the system would provide is a combined MATE and CEA model, where the tradespace plot (see FIG. 2) reports the performance of all feasible combinations of options (see Table 1 below) where at least one axis incorporates a measure of health-related outcomes, especially a metric emerging from a CEA, such as QALYs.

TABLE 1
Matrix of Options for a Drug Delivery System.

| Decision | Option 1 | Option 2 | Option 3 |
|---|---|---|---|
| Portability | IV pole attachment | Tabletop | On-body wearable |
| Administration speed | Impeller pump | Spring + syringe | Peristaltic pump |
| Mobile phone control | No | Yes | |

In addition to system architectural design options, clinical development and other strategic options would be intermingled into the set of decision-options. For example, including a decision-option of whether to pursue a longitudinal study of a new method to measure patient outcomes could be reflected in the tradespace plot, or enabled/disabled to explore the potential impact of the clinical development option, with consideration of its associated uncertainties. Or, for example, the assumed patient population or their patient journey could be adjusted to explore the impact on the model results, revealing a target system architecture and first indication with superior reimbursement potential.

Moreover, and as previously discussed, it is possible to combine multiple single-attribute utility metrics into a weighted multi-attribute utility metric. There is a technique called Multi-Criteria Decision Analysis (MCDA) which is used in MedTech reimbursement strategy and decision-making. The weights are scientifically determined through patient interviews, often in a process called Discrete Choice Experiments (DCE). According to some embodiments, it is possible to posit relationships between the decision-options and their impact on Patient-Reported Outcomes (PROs) as well as hypothesize weights that represent patient preferences for such PROs. That way, the MedTech value strategy can consider the relative value of challenging those underlying assumptions and reducing the uncertainty of those weights, and the hypothesized data can eventually be replaced with real data measured over time, which means the model can be a living digital twin (i.e., a “Value Twin”) that models and assesses value of the chosen path and threats in real time.

In more general terms, it is possible to anticipate patient preference information (PPI) into aggregate measures of value (e.g., as with MCDA), and thereby model the potential for treatment adoption. An example where PPIs may signal adoption more heavily than outcomes is something like end of life care or coma recovery, where some patients (or their families) may be driven by preferences that are at odds with life extension, for example.

In some embodiments, it is appreciated that the use or incorporation of clinical measures of reimbursable clinical effectiveness (as in cost-effectiveness or weighted PROs) as one of the axes in the plot is a novel aspect as is the framework and system that is provided to analyze these features. Below are example implementations of aspects of the method and system in particular verticals and problem spaces.

### Embodiment Verticals

The following example verticals show some practical illustrations on how some embodiments may be applied.

### Technology Researchers, Clinical Scientists, and Research Sponsors

Stewards of emerging technology can use this framework and system to identify health topics that warrant greater attention (for example, the highest margin between the standard of care and “utopia” point), or to understand how their projects compare to alternative research topics, either by including alternative technologies or alternative target patient populations. Commercially oriented researchers (for example, seeking to understand whether an invention warrants patenting, seeking startup funding, etc.) may use the proposed method to identify opportunities and threats to their investments and ventures. Clinical Scientists may wish to understand the clinical potential of emerging technologies (for example, by rapidly generating concepts to address a multitude of unmet needs). Steps:

1. Characterize the standard of care in terms of options to patients, including roadmapping the standard of care (past, present, forecasted, and potential disruptions). This may involve using clinical modeling best practices and expertise to interpret clinical data, physician input, and other assumptions and heuristics into a working and validated CEA model for each health topic. Repeat for as many patient populations, decision points, and variations within the standard of care as needed.
2. As applicable, represent a variety of competing existing and emerging technologies in terms of its solution-neutral (conceptual) function, which may include architectural variants and combinations of technologies. As needed, represent a given technology in solution-neutral terms to observe the impact across a multitude of clinical decision situations, with both discrete and aggregate measures of value (e.g. by multiplying reimbursable value by patient population size).
3. Assume relationships between each variant (option) and the impacted health-related outcome, for example the relative influences on CEA model parameters that impact QALYs. As applicable, calculate aggregate “value” (e.g. by multiplying reimbursable value by patient population size).
4. Characterize, quantify, and plot the uncertainty associated with each parameter relative to each variant (concept). This leads to probabilistic MATE. The benefit and usefulness of a probabilistic analysis is that it allows you to:
a. Combine divergent expert opinions (experts disagree) into a single analysis.
b. Identify the clinical value associated with the uncertainties (through sensitivity analysis), which allows the researcher or research sponsor to apply resources to reduce uncertainty in a way that supports future strategic decisions, too.
c. Uncertainty often correlates with financial valuation of a venture, so by understanding uncertainty, better assumptions can be made about the commercial potential of emerging technologies and the relative increase in valuation when that uncertainty is reduced. Research targets can therefore be shaped around these specific sources of uncertainty, which is useful to both researcher and sponsor.
5. Visually compare normalized (e.g., in terms of QALYs) evaluation of alternatives on an n-dimensional plot. Additionally, it is common practice in MATE to indicate a “Utopia” point, which often assumes perfect value, zero cost, zero side effects, etc. However, in some embodiments, the Utopia point is explicitly calculated and is an important strategic signal. Unlike many MATE studies, many CEA studies do not assume a perfect score (i.e., 1 QALY per year) even when the treatment works without side effects. Therefore, plotting an explicitly calculated Utopia point in a MATE tradespace plot is useful when comparing alternatives because it shows how much room there is to grow in the standard of care, and thus how much value can be created through technology research and development.

### MedTech Venture Capital

MedTech investment decision-makers analyze the potential value (payoff), timing of the payoff, and probability of the payoff, for example to calculate expected net present value (eNPV). The present invention supports these analyses to the degree that: payoff is associated with clinical performance of the technology; exploring uncertainties in the MATE and CEA models support understanding the probability of payoffs; and, evaluating strategic and architectural alternatives in the MATE model permits evaluation of additional qualities like time to value, risk, regulatory success, clinical adoption, policy uncertainty, etc. Additionally, the analysis can be extended to include competing medical technologies to identify threats.

Valuation of a MedTech startup at exit depends heavily on the pre-clinical and clinical data already produced that may give assurance of the success of the venture. Therefore, a VC may use the aspects to:

1. Survey the landscape of the indication (disease state targeted by the treatment) for available and potentially competing treatments in the standard of care.
2. Clarify assumptions about how the technology works as a generalized concept, where each function is assigned a probability. Assigning a probability appropriately suggests risk and that reducing uncertainty through technology and/or clinical development builds venture value.
3. Model assumptions as probability distributions, which helps to identify critical assumptions and integrate potentially optimistic founder opinions with divergent expert opinions (optimistic or pessimistic).
4. Identify the key assumptions and sources of uncertainty that can be challenged and/or eliminated and prioritize them according to their strategic value, as may be indicated in the probabilistic analysis of tradeoffs.
5. More effectively mentor the venture founders, executives, and board members and/or create contractual obligations or milestones based on founders' commitment to and creation of strategic value, as made possible through the analysis.
6. Enable enhanced alignment around company vision and opportunity, leading to enhanced relationships, utilization of resources, recruiting, and compensation.
7. Enable enhanced learning through the process of explicitly defining model structure, parametric assumptions, variables, and probabilistic distributions.
8. Support investment go/no-go decisions with holistic valuation of the venture, including its threats, as well as follow-on investments, which is useful for platform technologies.

### Venture Founders Steering R&D, Pre-Clinical or Clinical Development, and Operations

The details of the embodiment vertical related to venture capital investors also apply to venture founders as an embodiment vertical, with the added benefit of aligning operations around the strategic insights, including assisting in the development of corporate knowledge, hiring vs. outsourcing decisions, build vs. buy decisions, conflict resolution activities, defending the venture from threats, and enhancing team motivation and cohesion. Venture founders and would-be founders, like investors, wish to understand the key sources of and threats to value creation. An analysis of tradeoffs can highlight the relationship between uncertainty and strategic value.

Similar to how a VC may use aspects, a venture founder may use certain embodiments to:
1. Develop explicit assumptions about the mechanisms that drive treatment effectiveness. For example, is the treatment a complete fix, or only a partial fix? Does it only address symptoms? Do some aspects of the technology treat disease while other aspects reduce side effects?
2. Characterize the effectiveness of other treatments, too. It is also possible to compare and contrast treatments in other ways, for example:
Duration of treatment. For example, single dose or multiple attempts required?
Likelihood of adverse events. For example, infection from an implant.
Patient tolerance of side-effects.
Patient preferences, such as trade-offs in risk and effectiveness
   3. Identify other attributes that may influence the analysis. For example, how does the patient disease state present itself and in what clinical decision situations? How many clinical decision points are there for a given population with the disease? How diverse or dispersed are they? Is there some centrality and/or cohesiveness to the power of decision-making? Is there some interdependence to clinical decisions? Would disruption be costly and/or difficult?
   4. With consideration of tradeoffs and uncertainty, identify opportunities to create strategic value, and then construct a preclinical or clinical development plan that maximizes strategic value.

### Drug Delivery and Combination Product Valuation and Development

For biopharmaceutical assets, in some implementations clinical development must show that the drug or biologic provides the target anticipated value and strategic positioning. Often, pairing the drug with a delivery device (or administering the drug as part of a combination product) is proposed to enhance and protect the value of the treatment and create other opportunities to deliver value. For example, an “artificial organ” can sense patient biomarkers and administer the drug, leading to higher adherence and better health outcomes, and patients may report higher quality of life.

The proposed method can be used to weigh the attributes of design alternatives. For example, the size and portability of the drug delivery system, the number of steps required to refill/begin an administration, or the amount of control given to the patient can all be explicitly shown as tradeoffs in overall clinical value, for example.

The proposed method can also be used to compare innovation initiatives based on their potential impact to the standard of care. For example, an implantable pump may be improved by improving miniaturization, form factor, battery life, materials, connectedness, control algorithms, safety features, and/or refilling capabilities. Each potential area of improvement comes with risks and benefits, which can be normalized as high-level metrics of value (e.g. QALYs, PROs, etc.). This example illustrates how certain embodiments can help to further position biopharmaceutical assets (e.g., drugs, biologics, biotechnologies, etc.) through the combination product development process (e.g., by selecting an accompanying drug delivery system). certain embodiments, when applied, may influence holistic strategies around capabilities, ecosystem development, vendor relationships, etc.

### Cross-Functional Biopharma Development Teams

Cross-functional team members represent different and often competing interests in biopharma development projects. For example, the optimal paths to maximize reimbursement may be in tension with the most efficient clinical trial options, or the lowest-risk paths to regulatory approval. The present invention can be tailored to represent the specific needs and/or concerns of cross-functional development team members as measures of ‘value’ in the tradespace analysis. Doing so can enhance discussions, enable negotiations, and break down silos by showing team members the zone of potential agreement (ZOPA) and revealing tradeable parameters.

### Digital Health Solutions

When development options for digital health solutions are evaluated using aspects of the present invention, it will be possible to include options like the use of wearables, remote patient monitoring solutions, apps, connected drug delivery devices, AI/ML algorithms, etc., so that options and combinations of options can be evaluated in terms of their potential value and costs. This enables system architecture analysis and decisions with a closer link to value.

Regulatory Strategy: 510(k) vs. PMA

A common decision point in medical device development includes determining whether to request FDA clearance through the 510(k) process or with a Pre-Market Authorization (PMA). A 510(k) can require much less time and effort because it assumes the proposed device is “substantially equivalent” to a device that is already cleared to market. A PMA is required when there is no substantial equivalent, and it may be necessary to show through clinical studies that the device provides the benefit(s) claimed by the manufacturer. Though a 510(k) can be more expeditious, it is sometimes better to do a PMA when gathering evidence of clinical value leads to higher adoption rates and reimbursement approvals.

When considering this important and strategic decision of whether to go through a 510(k) or a PMA, some approaches described herein can be used to obtain an early-stage clinical comparison (i.e., with CEA) of the substantially equivalent device to the new device, and fundamental sources of uncertainty can be identified and targeted as potential sources of value, to allow for a more adaptive development strategy. In this way, using the analysis to enhance learning about how uncertainty drives clinical value can allow for a flexible approach, i.e., that the venture can abandon the PMA path if the clinical value does not materialize.

### Technology Scouting & Market Opportunity Estimation

In some embodiments, the system enables modeling the standard of care in terms of available, known-to-be-emerging, and anticipated treatment options. The system may model the standard of care at multiple points in time, including forecasted future time points. As with all other model structural features, elements, attributes, parameters, and constants, the user may influence and alter the model, as applicable, to improve forecasts or to explore “what-if” scenarios, for example. For example, expiring IP may influence the cost-effectiveness of a currently available treatment option at a future time point, and these details can be built into the model at future timepoints. The process of identifying patterns and trends, and developing forecasts, may be referred to as roadmapping, and so the present invention may be used for roadmapping the standard of care.

In some embodiments, the system may search broadly for emerging technologies such that standard of care roadmap may include emerging technologies. Even if no clear scientific rationale or initial data are available to indicate clinical cost-effectiveness, the model may reflect the emerging technology in terms of its generalized function, whether at the component or system level, and its relative effectiveness as an uncertainty distribution with essentially unbounded uncertainty. The model may also include intermediate metrics of value—for example, factors that should be optimized in an emerging technology to increase its utility (e.g., battery life, form factor, etc.). Ultimately, relationships are posited between the underlying technological assumptions and their impact on overall value, as modeled. The user may influence or alter the model, too.

Emerging options in the standard of care, as modeled, may also be combined with an enumeration of the available treatment R&D decisions. When relationships are posited between the available R&D decisions and their potential to impact the standard of care, the model may communicate the potential impact of R&D decisions on value, such as, for example, cost-effectiveness.

In some embodiments, the system further supports the technology scouting and market estimation processes because clinical cost-effectiveness studies are often based on population studies, which means the value in a specific modeled clinical scenario can be extrapolated across the patient population size to compare options not just on their individual impact but in terms of broader economic value. When combined with a willingness-to-pay (WTP) assumption (which can be probabilistic, too), this example of extrapolating from individual clinical value to population value provides an estimate of the Total Addressable Market (TAM) because it identifies unmet needs at the level of the healthcare market.

### Technology Roadmapping

Technology roadmapping is an emerging practice in MedTech portfolio management that involves characterizing a technology in terms of where the technology came from and where the technology could go, for example. Roadmapping can be a formally structured process—for example, see the Advanced Technology Roadmap Architecture (ATRA) framework. Generally, like MATE, the ATRA framework involves abstracting the technology into a solution-neutral function and/or embodiment and characterizing its development against solution-neutral metrics. For example, technology roadmapping in aviation would observe how embodiments evolved along solution-neutral metrics like speed, range, payload capacity, comfort, and fuel efficiency.

Technology roadmapping is difficult in MedTech because medicine is complex. According to some embodiments described herein, this approach enables roadmapping in MedTech because it involves describing solution-neutral performance in terms of overall clinical effectiveness metrics (e.g., QALYs). It is then helpful to understand the historical record of clinical performance for different technologies, as well as forecast changes in the standard of care, economics, or any other factors that may affect patient outcomes in a patient population of interest.

To develop a roadmap in terms of clinical effectiveness rather than an intermediate metric (e.g., size, weight, number of doses, number of steps, etc.) is novel and unique. It is also useful for keeping value in mind and monitoring for potential threats (e.g., in real time).

Because roadmapping is time-dependent, there is additional value in an all-in-one view of the competitive landscape because the competitive landscape can be animated to include time-variant aspects like the emergence of new technologies, expiration of patents, etc.

### Care Providers Architecting Treatment Algorithms

As care providers centralize (e.g., with the emergence of Accountable Care Organizations (ACOs) in a Value-Based Care system), providers make treatment algorithm decisions that impact large populations of patients. Their investments in technologies, capabilities, and staff become more targeted, and therefore more strategic with the goal of improving outcomes for a patient population. This method can help providers (like ACOs) identify and compare treatment options (or combinations of treatment options) in terms of their benefit to patients, as well as against other tradeoffs of interest.

### Wraparound Services

Wraparound services, when evaluated in terms of their impact on clinical value, can be included in the analysis. Because various embodiments described herein enable combinations of systems options, different services concepts can be combined with any number of other services or treatment options to compare the relative costs and benefits of different options and policies in a tradespace analysis.

### Design for Risk Management

The proposed method shows trade-offs, which can be adapted to show trade-offs between risk-related aspects of a treatment. For example, robotic surgery reduces invasiveness, but early implementations saw higher error rates due to the need for highly trained operators. It is possible to represent these risks as impacts to overall clinical effectiveness (e.g., as deducted QALYs) to more holistically explore the value potential of emerging technologies.

The best practices for risk management in industry suggest that the preferred way to reduce risk is in the following order:
1. Elimination
2. Reduction
3. Protective measures
4. Information

However, it is far more common for risk management practices to begin after the concept has already been designed, precluding opportunities to revisit Option 1 (Elimination). This method can be used to weigh the risk-level of design alternatives in terms of overall clinical effectiveness, which adds a new dimension of awareness in risk management processes. Normalizing options in terms of clinical effectiveness assures that the final product will have an optimal clinical risk-benefit rationale, and may also impact reimbursement and adoption (e.g., by a large hospital system). This approach can help to focus the otherwise complicated and information-rich process of risk-benefit analysis in medical technology development, and can support related regulatory filings, medical documentation, etc., for example by showing how early estimates of risk were reduced in development and validation activities.

### Reimbursement Decision-Making

While there is a growing interest in using clinical effectiveness metrics (e.g., QALYs) to measure the impact of a treatment, there are other factors that impact reimbursement. The ISPOR ‘Value Flower’ mentions Equity, Value of Hope, and Reduction in Uncertainty, to name a few. It is therefore helpful to see both QALYs alongside these and other metrics (e.g., patient-reported outcomes, patient preferences, adherence, etc.). The tradespace plot is often used to indicate multi-attribute utility by combining multiple measures of value into a single unitless metric. This is consistent with the practice of Multi-Criteria Decision Analysis (MCDA), which is essentially a weighted sum of reported outcomes that can signal overall treatment value. This practice can be extended to incorporate any measure or combination of measures of health-related value, which can be used to identify dominant and dominated treatment options, as well as to conduct what-if analyses to reveal the assumptions or conditions under which the results of the analysis may change.

This practice is valuable because explicitly defining value and costs across different measures of value (e.g., as axes) gives a visual indication of value positioning (e.g., as with a biopharmaceutical). Combined with the inclusion of uncertainty, the value and risk of evidence gathering activities can be estimated as well. For instance, when the development team has already planned an evidence generation activity, the analysis can help to identify risks and gain clarity on the relative value that is expected when the activity is complete. The options and competing alternatives can be represented in n-dimensional tradespace plots as nominal points and/or as uncertainty distributions.

The system according to various embodiments permit the user to manually or automatically toggle and select the axes and apply groupings (of metrics, with or without weights) to show positioning, including from the perspective of stakeholders like prescribers, patients (with consideration of their values), payers, hospital administrators, caregivers, communities, society, and more. These ‘personas’ can be manually or automatically scoped.

In further embodiments described in Appendix A, the methods and solutions provided therein may be used alone or in combination with the embodiments described here, and it should be appreciated that Appendix A forms an integral part of the instant application.

### Biopharma Portfolio Management

When all company assets are evaluated, it is possible to elicit portfolio-level insights, such as how assets are both differentiated and similar to one another. Overlap between value proposition can be visually shown in the tradespace plot, and derivative plots such as when potential value is plotted against risk, which can be a metric in the analysis.

Biopharma companies using GenAI will find aspects described herein to be a useful “sidecar” to the existing process, which can generate more candidate drugs than the industry can progress due to the high cost, risk, uncertainty, and duration of development for a single drug. An example is gene therapy, where a platform technology may be extended/targeted to any number of hereditary diseases, but other factors like the delivery method and scientific risk come with a lot of uncertainty.

### CONCLUSION AND IMPLEMENTATIONS

Various examples are methods that can be implemented either on a single or a combination of computer-based systems in a distributed network. Method examples are completed in various locations and by one or more systems. For example, and in accordance with the various aspects and embodiments, IP elements or units include processors (e.g., CPUs, GPUs, or NPUs), random-access memory (RAM—e.g., off-chip dynamic RAM or DRAM), a network interface for wired or wireless connections such as Ethernet, WIFI, 3G, 4G long-term evolution (LTE), 5G, 6G and other wireless interface standard radios. The system may also include various I/O interface devices, as needed for different peripheral devices such as touch screen sensors, geolocation receivers, microphones, speakers, Bluetooth peripherals, and USB devices, such as keyboards and mice, among others. By executing instructions stored in RAM devices processors perform steps of methods as described herein.

Some examples are one or more non-transitory computer readable media arranged to store such instructions for methods described herein. Whatever machine holds non-transitory computer readable media comprising any of the necessary code may implement an example. Some examples may be implemented as: physical devices such as semiconductor chips; hardware description language representations of the logical or functional behavior of such devices; and one or more non-transitory computer readable media arranged to store such hardware description language representations. Descriptions herein reciting principles, aspects, and embodiments encompass both structural and functional equivalents thereof. Elements described herein as coupled have an effectual relationship realizable by a direct connection or indirectly with one or more other intervening elements.

Some embodiments may involve or include one or more machine learning models. In some aspects, the systems and methods described herein may utilize one or more machine learning models to perform various functions and operations. These machine learning models may be implemented on one or more computer systems, which may include local computing devices, remote servers, cloud-based computing platforms, or distributed computing environments. The machine learning models may be trained using various techniques and may be configured to process input data and generate output predictions, classifications, or other results based on learned patterns and relationships.

In some cases, the one or more machine learning models may include supervised learning models, unsupervised learning models, semi-supervised learning models, or reinforcement learning models. The machine learning models may comprise neural networks, decision trees, support vector machines, random forests, gradient boosting machines, or other types of machine learning architectures. In some aspects, the neural networks may include deep learning models such as convolutional neural networks, recurrent neural networks, transformer models, or generative adversarial networks.

The one or more computer systems on which the machine learning models operate may include processors, memory, storage devices, and network interfaces configured to execute the machine learning models and process data. In some aspects, the computer systems may include specialized hardware such as graphics processing units (GPUs), tensor processing units (TPUs), or field-programmable gate arrays (FPGAs) to accelerate machine learning computations. The computer systems may be configured to receive input data from various sources, process the data using the machine learning models, and provide output results to users or other systems.

In some cases, the machine learning models may be trained using training data that includes labeled examples, unlabeled examples, or a combination thereof. The training process may involve adjusting parameters of the machine learning models to minimize a loss function or maximize a performance metric. Once trained, the machine learning models may be deployed on the one or more computer systems to perform inference operations on new input data. The machine learning models may be periodically retrained or updated based on new data or changing conditions to maintain or improve their performance over time.

Practitioners skilled in the art will recognize many modifications and variations. The modifications and variations include any relevant combination of the disclosed features. Descriptions herein reciting principles, aspects, and embodiments encompass both structural and functional equivalents thereof. Elements described herein as “coupled” or “communicatively coupled” have an effectual relationship realizable by a direct connection or indirect connection, which uses one or more other intervening elements. Embodiments described herein as “communicating” or “in communication with” another device, module, or elements include any form of communication or link and include an effectual relationship. For example, a communication link may be established using a wired connection, wireless protocols, or other communication methods.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

When introducing elements of the present disclosure or the embodiments thereof the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although some embodiments have been described with a certain degree of particularity, it is to be understood that the present disclosure has been made only by way of illustration and that numerous changes in the details of construction and arrangement of parts may be resorted to without departing from the spirit and the scope of the invention.

1.-42. (canceled)

43. An apparatus for supporting decision-making in medical technology strategy, comprising:
a Multi-Attribute Tradespace Exploration (MATE) module configured to generate system architectural decision-options and plot them in a tradespace plot;
a Clinical Cost-Effectiveness Analysis (CEA) module configured to calculate clinical effectiveness metrics, including measures of quality of life and years of survival (e.g., quality-adjusted life years (QALYs), disability adjusted life years (DALYs), and equivalent value of life-years gained (evLYGs)), for the decision-options (including combinations of decision-options);
a relationship positing module configured to establish relationships between the decision-options and their impact on clinical effectiveness;
a probabilistic analysis module configured to perform Monte Carlo simulations to account for uncertainty in performance of the decision-options;
a visualization module configured to provide a coordinated visualization of the decision-options in terms of multiple dimensions of value, their uncertainty, and attributes of other treatment options;
wherein the apparatus integrates outputs of the MATE module and the CEA module to generate a combined model that supports decision-making in medical technology strategy.

44. The apparatus according to claim 43, wherein the MATE module is further configured to evaluate trade-offs between different architectural decision-options based on multiple criteria including cost, performance, and risk.

45. The apparatus according to claim 44, wherein the multiple criteria includes one or more measures of utility including multi-attribute utilities.

46. The apparatus according to claim 44, wherein the CEA module is further configured to incorporate patient-reported outcomes in a calculation of at least one of a group comprising clinical effectiveness metrics, other health-related metrics, and multi-criteria measures of treatment performance or value.

47. The apparatus according to claim 46, wherein the relationship positing module is further configured to use one or more machine learning algorithms to establish relationships between a function of a technology and its impact on clinical effectiveness.

48. The apparatus according to claim 47, wherein the function of the technology includes one or more of the decision-options that are provided as inputs to the one or more machine learning algorithms.

49. The apparatus according to claim 48, wherein the probabilistic analysis module is further configured to perform sensitivity analysis to identify one or more most influential variables affecting outcomes of the decision-options.

50. The apparatus according to claim 49, wherein the probabilistic analysis module is further configured to perform one-way sensitivity analysis on a group of variables to identify one or more influential variables.

51. The apparatus according to claim 50, wherein the probabilistic analysis module is further configured to replace one or more influential variables with probabilistic distributions.

52. The apparatus according to claim 49, wherein the probabilistic analysis module is further configured to perform probabilistic sensitivity analysis.

53. The apparatus according to claim 49, wherein the probabilistic analysis module is further configured to output, to a display, probabilistic performance relative to other decision-options or existing treatments in a standard of care.

54. The apparatus according to claim 49, wherein the visualization module is further configured to generate interactive visualizations that allow users to explore different scenarios and their potential impacts on clinical effectiveness.

55. The apparatus according to claim 54, wherein the combined model integrates real-time data inputs to continuously update a decision-making support framework.

56. The apparatus according to claim 55, wherein the apparatus is further configured to generate a report summarizing a set of design options and their associated clinical effectiveness metrics.

57. The apparatus according to claim 56, wherein the associated clinical effectiveness metrics include clinical effectiveness relative to a reference treatment, for example, a dominant treatment in a standard of care or anticipated competitive situation.

58. The apparatus according to claim 57, wherein the MATE module includes a feature for prioritizing decision-options based on user-defined criteria.

59. A non-transitory computer-readable medium storing instructions that, when executed by a processor, cause the processor to perform a method comprising:
modeling, in a computer system, a first model representing a plurality of decision-options, the plurality of decision-options including an associated cost;
modeling, in the computer system, a second model representing an effectiveness of the plurality of decision-options;
optimizing, by the computer system, the plurality of decision-options of the first model and the effectiveness of the plurality of decision-options of the second model; and
providing an output indicating performance of a set of design options selected from the plurality of decision-options against a plurality of metrics.
